(19) United States
(12) Patent Application Publication
(10) Pub. No.: US 2021/0038162 A1
(43) Pub. Date: Feb. 11, 2021

(54) METHOD, DEVICE, AND PROGRAM FOR ESTIMATING PULSE RATE

(71) Applicant: Nippon Telegraph and Telephone Corporation, Tokyo (JP)

(72) Inventors: Shin Toyota, Tokyo (JP); Shoichi Oshima, Tokyo (JP); Kenichi Matsunaga, Tokyo (JP)

(21) Appl. No.: 17/044,489

(22) PCT Filed: Mar. 27, 2019
(86) PCT No.: PCT/JP2019/013180

§ 371 Date: Oct. 1, 2020

(30) Foreign Application Priority Data
Apr. 4, 2018 (JP) 2018-072200

(51) Int. Cl. A61B 5/00; A61B 5/024

(52) U.S. Cl. CPC A61B 5/7221; A61B 5/02433; A61B 2562/0238; A61B 5/6817; A61B 5/7203

(57) ABSTRACT

A pulse wave is detected chronologically. Time-frequency analysis is performed on the detected pulse wave to determine a power spectrum for each time. Local maximum points are determined for each one of the power spectra determined for the respective times. For each one of the power spectra determined for the respective times, a certain number of largest values of the determined local maximum points are extracted as pulse rate candidates for that time. A pulse rate candidate is compared with a pulse rate candidate for the preceding time to determine a difference in frequency between them, and pulse rates for which the determined difference in frequency is equal to or greater than a preset reference value are eliminated from the candidates.

### CROSS-REFERENCE TO RELATED APPLICATIONS

This application is a national phase entry based PCT Application No. PCT/JP2019/013180, filed on Mar. 27, 2019 which claims priority to Japanese Application No. 2018-072200, filed on Apr. 4, 2018, which applications are hereby incorporated herein by reference.

### TECHNICAL FIELD

The present invention relates to a pulse rate estimation method and apparatus and a program for determining a pulse rate from a detected pulse wave.

### BACKGROUND

For measurement of variations in a pulse wave over a long period of time, pulse wave diagnostic devices that optically detect a pulse wave are used (see Patent Literature 1). The pulse wave diagnostic device of Patent Literature 1 receives transmitted light that has been transmitted through an artery or scattered light that has been scattered by an artery to detect a pulse wave, calculates a pulse wave amplitude for each pulse of the detected pulse wave, and calculates a point of pulse wave amplitude on a rectangular coordinate plane formed by two pulse wave amplitudes that were consecutively calculated as a Poincare coordinate for each single pulse. This technique enables detection of variations in a pulse wave over a long time period because it does not involve electrodes to be attached on skin surface.

### CITATION LIST

### Patent Literature

Patent Literature 1: Japanese Patent No. 5160586.

### SUMMARY

### Technical Problem

The aforementioned technique, however, has a disadvantage of being unable to determine an accurate pulse rate when components originating from body motion are present in the detected signal because it presupposes that variations in a detected signal (pulse wave) are pulse wave components alone.

Embodiments of the present invention have been made in order to solve such a disadvantage and has an object of enabling more accurate determination of a pulse rate from a detected pulse wave.

### Means for Solving the Problem

A pulse rate estimation method according to embodiments of the present invention includes: a first step of chronologically detecting a pulse wave; a second step of performing time-frequency analysis on the detected pulse wave to determine a power spectrum for each time; a third step of determining local maximum points in each one of the power spectra determined for respective times; a fourth step of extracting, for each one of the power spectra determined for the respective times, a certain number of largest values of the determined local maximum points as pulse rate candidates for that time; and a fifth step of comparing a pulse rate candidate with a pulse rate candidate for a preceding time to determine a difference in frequency between them, and eliminating pulse rates for which the determined difference in frequency is equal to or greater than a preset reference value from the candidates.

The pulse rate estimation method may further include a sixth step of determining, after the fifth step, a candidate having a highest local maximum at each time as an actual pulse rate.

The pulse rate estimation method may further include a seventh step of reducing noise in the pulse wave detected in the first step, and in the second step, the pulse wave with reduced noise may be subjected to time-frequency analysis.

A program according to embodiments of the present invention is a program for causing a computer to execute the pulse rate estimation method set forth above.

A pulse rate estimation apparatus according to embodiments of the present invention includes: a detection unit that chronologically detects a pulse wave by receiving transmitted light that has been transmitted through an artery or scattered light that has been scattered by an artery; a first processing unit that performs time-frequency analysis on the pulse wave detected by the detection unit to determine a power spectrum for each time; a second processing unit that determines local maximum points in each one of the power spectra determined by the first processing unit for respective times; a third processing unit that extracts, for each one of the power spectra determined for the respective times, a certain number of largest values of the local maximum points determined by the second processing unit as pulse rate candidates for that time; and a fourth processing unit that compares a pulse rate candidate extracted by the third processing unit with a pulse rate candidate for a preceding time to determine a difference in frequency between them, and eliminates pulse rates for which the determined difference in frequency is equal to or greater than a preset reference value from the candidates.

The pulse rate estimation apparatus may further include a fifth processing unit that determines, after processing at the fourth processing unit, a candidate having a highest local maximum at each time as an actual pulse rate.

The pulse rate estimation apparatus may further include a filter unit that reduces noise in the pulse wave detected by the detection unit, and the first processing unit may perform time-frequency analysis on the pulse wave with noise reduced by the filter unit.

### Effects of Embodiments of the Invention

With the foregoing, embodiments of the present invention provide an advantageous effect of being able to determine a pulse rate more accurately from an optically detected pulse wave.

### BRIEF DESCRIPTION OF THE DRAWINGS

FIG. 1 is a flowchart for describing a pulse rate estimation method in an embodiment of the present invention.

FIG. 2 is a configuration diagram showing a configuration of a pulse rate estimation apparatus in an embodiment of the present invention.

FIG. 3 is a configuration diagram showing a hardware configuration of the pulse rate estimation apparatus according to the present invention.

FIG. 4 is a configuration diagram showing a further configuration of the pulse rate estimation apparatus in an embodiment of the present invention.

FIG. 5 is a configuration diagram showing a further configuration of the pulse rate estimation apparatus in an embodiment of the present invention.

FIG. 6 is a flowchart for describing the pulse rate estimation method in an embodiment of the present invention in more detail.

FIG. 7 shows a spectrogram of a signal (pulse wave) observed over a certain time period.

FIG. 8 shows a power spectrum at a certain time taken from spectrogram shown in FIG. 7.

FIG. 9 shows an explanatory diagram that illustrates a result of estimating the pulse rate on the spectrogram illustrated in FIG. 7.

FIG. 10 is a flowchart for describing another pulse rate estimation method in an embodiment of the present invention in more detail.

### DETAILED DESCRIPTION OF ILLUSTRATIVE EMBODIMENTS

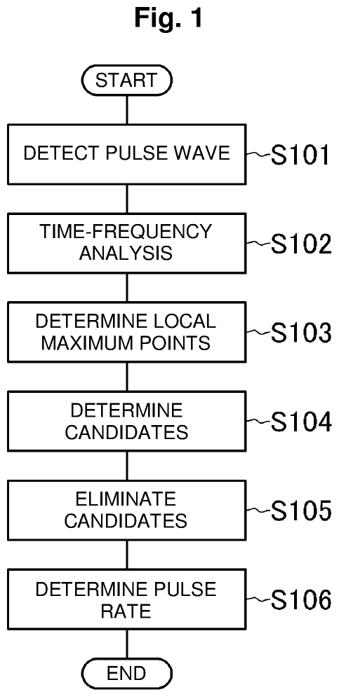

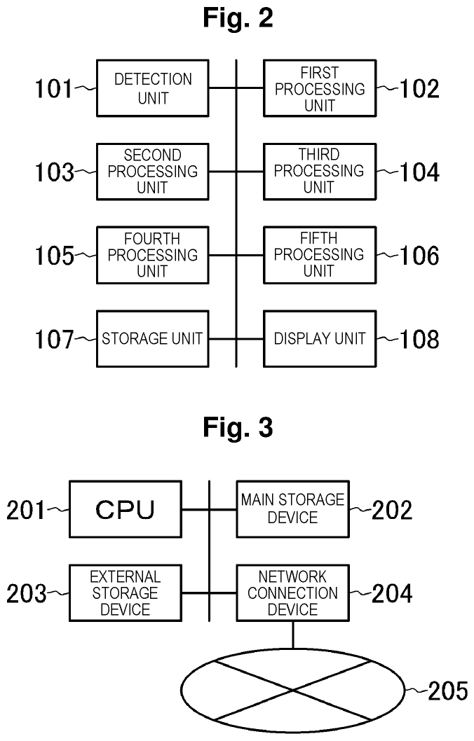

A pulse rate estimation method in an embodiment of the present invention is now described with reference to FIG. 1. First, in step S101, a pulse wave is detected chronologically (a first step). For example, the pulse wave may be detected by receiving transmitted light that has been transmitted through an artery or scattered light that has been scattered by an artery. Then, in step S102, time-frequency analysis is performed on the detected pulse wave to determine a power spectrum for each time (a second step).

Then in step S103, local maximum points are determined for each one of the power spectra determined for the respective times (a third step). Next, in step S104, for each one of the power spectra determined for the respective times, a certain number of largest values of the determined local maximum points are extracted as pulse rate candidates for that time (a fourth step).

Then, in step S105, a pulse rate candidate is compared with a pulse rate candidate for the preceding time to determine a difference in frequency between them, and pulse rates for which the determined difference in frequency is equal to or greater than a preset reference value are eliminated from the candidates (a fifth step).

In this embodiment, in step S106 following step S105, the candidate having the highest local maximum at each time is also determined as the actual pulse rate (a sixth step). Optionally, noise may be reduced in the pulse wave detected in step S101 (a seventh step), after which the pulse wave with reduced noise may be subjected to time-frequency analysis in step S102.

A pulse rate estimation apparatus in an embodiment of the present invention is now described. The pulse rate estimation apparatus includes a detection unit 101, a first processing unit 102, a second processing unit 103, a third processing unit 104, a fourth processing unit 105, a fifth processing unit 106, a storage unit 107, and a display unit 108.

The detection unit 101 chronologically detects a pulse wave. For example, the detection unit 101 detects the pulse wave by receiving transmitted light that has been transmitted through an artery or scattered light that has been scattered by an artery. The first processing unit 102 performs time-frequency analysis on the pulse wave detected by the detection unit 101 to determine a power spectrum for each time. The second processing unit 103 determines local maximum points in each one of the power spectra determined by the first processing unit 102 for the respective times.

The third processing unit 104 extracts, for each one of the power spectra determined for the respective times, a certain number of largest values of the local maximum points determined by the second processing unit 103 as pulse rate candidates for that time. The pulse rate candidates are stored in the storage unit 107, for example. The fourth processing unit 105 compares a pulse rate candidate extracted by the third processing unit 104 with a pulse rate candidate for the preceding time to determine a difference in frequency between them, and eliminates pulse rates for which the determined difference in frequency is equal to or greater than a preset reference value from the candidates.

After processing at the fourth processing unit 105, the fifth processing unit 106 determines the candidate having the highest local maximum at each time as the actual pulse rate, and displays the pulse rate on the display unit 108, for example. Optionally, a filter unit (not shown) that reduces noise in the pulse wave detected by the detection unit 101 may be included. In that case, the first processing unit 102 may perform time-frequency analysis on the pulse wave with noise reduced by the filter unit.

The pulse rate estimation apparatus in the above embodiment is a computer device including a CPU (Central Processing Unit) 201, a main storage device 202, an external storage device 203, and a network connection device 204 as shown in FIG. 3, where the functions described above are implemented by the CPU 201 operating according to a program loaded in the main storage device for executing the pulse rate estimation method. The network connection device 204 connects to a network 205. The functions may be distributed among multiple computer devices.

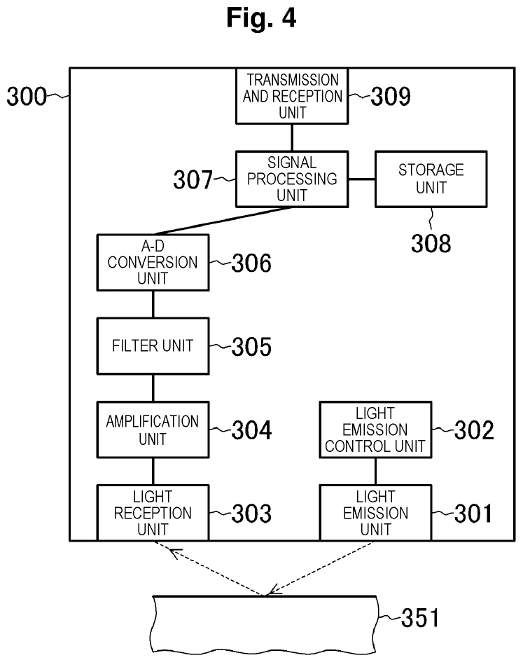

More detailed descriptions are provided below. For example, as shown in FIG. 4, a pulse rate estimation apparatus 300 may include a light emission unit 301, a light emission control unit 302, a light reception unit 303, an amplification unit 304, a filter unit 305, an A-D conversion unit 306, a signal processing unit 307, a storage unit 308, and a transmission and reception unit 309.

The light emission unit 301 is composed of a light emitting element, e.g., light emitting diode or semiconductor laser, and radiates light of a predetermined wavelength (infrared) to a site of measurement 351 under the control of the light emission control unit 302. The light emission unit 301 may be composed of one or more light emitting elements. The light emitted by the light emission unit 301 is transmitted through skin at the site of measurement 351 to irradiate blood in peripheral arteries.

The light that has been radiated by the light emission unit 301 and returned from the site of measurement 351 is received by the light reception unit 303 and goes through photoelectric conversion. The light reception unit 303 corresponds to the detection unit described above. The light reception unit 303 is composed of one or more light reception elements such as photodiodes, receives light that has been incident on the site of measurement 351 and has been transmitted through an artery and light scatted by an artery, and converts it to an analog signal.

The analog signal resulting from the photoelectric conversion at the light reception unit 303 is amplified by the amplification unit 304 to a predetermined signal level suited for signal processing, and particular frequency components are extracted from it at the filter unit 305. The filter unit 305 passes signal components of frequency bands in a range in which a heart rate (pulse rate) varies, e.g., 0.7 Hz to 4.0 Hz.

The analog signal with the particular frequency components extracted at the filter unit 305 is converted to a digital signal at the A-D conversion unit 306. The signal processing unit 307 estimates the pulse rate from this digital signal. The signal processing unit 307 corresponds to the first processing unit 102, the second processing unit 103, the third processing unit 104, the fourth processing unit 105, and the fifth processing unit 106 described above, being composed of a CPU and other components as mentioned earlier.

The signal processing unit 307 performs processing in accordance with a pulse rate estimation algorithm program, thus calculating the pulse rate of an observed person based on an observation signal acquired from the light reception unit 303. The signal processing unit 307 processes the digital signal in accordance with a stored program, saves the calculated pulse rate in the storage unit 308 and reads it therefrom. The storage unit 308 has a data saving area and a program saving area formed from non-volatile memory, a working area formed from volatile memory and the like, for example.

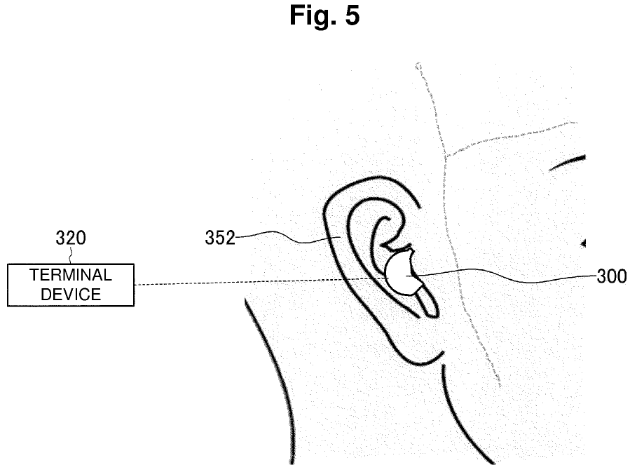

The signal processing unit 307 also outputs data such as the calculated pulse rate to the transmission and reception unit 309 for wireless communication. Here, the pulse rate estimation apparatus 300 has an appearance in the shape of an earphone for attachment to an ear 352 of a subject person, as illustrated in FIG. 5. In FIG. 5, the dotted lines indicate peripheral arteries. The pulse rate estimation apparatus 300 may be configured to wirelessly communicate with an external terminal device 320 via the transmission and reception unit 309 as shown in FIG. 5. For example, the transmission and reception unit 309 performs transmission and reception with the terminal device 320 and transmits data output from the signal processing unit 307 to the terminal device 320.

While the above description showed an arrangement for observing a photoplethysmographic pulse wave via the light emission unit 301 and the light reception unit 303, embodiments of the present invention are not limited to a photoplethysmographic pulse wave approach but observation signals may be acquired with a pressure pulse wave approach or an electrocardiographic heart rate approach.

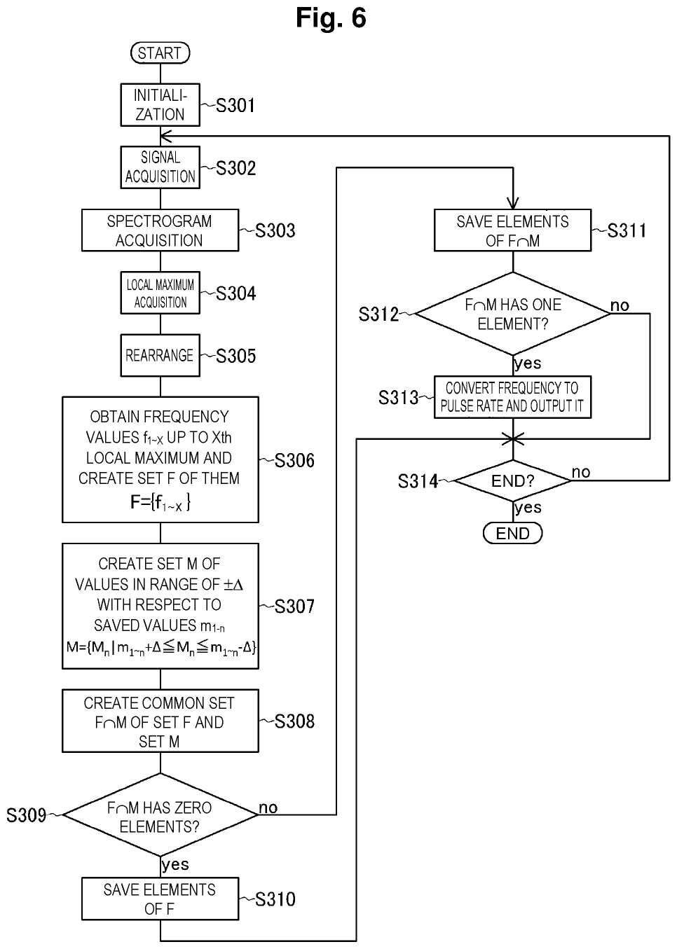

Referring now to FIG. 6, an algorithm for determining a pulse rate from frequency components with a spectrogram (the pulse rate estimation method) will be described. First, in step S301, working memories in the storage unit 308 are initialized. In this example, x=0 and m1=0 are substituted into m1 to m2 saved in working memories. Then, in step S302, a pulse wave signal for the observed person is acquired for a certain period of time.

Specifically, upon the start, a particular light emitting element of the light emission unit 301 is caused to emit light at a predetermined light emission intensity through control by the light emission control unit 302 to irradiate the site of measurement 351. The incident light is scatted by arteriole near the site of measurement 351 to exit the skin surface at the site of measurement 351 and received by the light reception element(s) of the light reception unit 303. An analog signal corresponding to the amount of light thus received by the light reception unit 303 is output. The output signal is then amplified by the amplification unit 304 and is output to the signal processing unit 307 as an observation signal via the filter unit 305 and the A-D conversion unit 306.

Then, in step S303, the signal processing unit 307 performs time-frequency expansion such as short-time Fourier transform and wavelet transform based on chronological data for the observation signal (spectrogram acquisition).

Then, in step S304, the signal processing unit 307 obtains the power spectrum at a certain time Th, determines local maximums in it by performing peak search on the obtained power spectrum, and records the intensity and frequency values of the local maximums. Specifically, a certain width is established on frequency axis with respect to a function of the obtained spectrum and local maximums are determined within the established width. Local maximums are determined while shifting the width and the intensity and frequency values of all the local maximums are recorded.

Then, in step S305, the signal processing unit 307 sorts the recorded local maximums according to the power value to rearrange them in descending order. Then, in step S306, the signal processing unit 307 extracts local maximums up to the Xth local maximum and records their frequency values f1˜X. Here, the set of f1˜X is represented as F, where F={fX|X being a natural number}. These frequency values are candidates for the pulse rate.

Then, in step S307, the signal processing unit 307 creates a set M of values that fall within a range of a certain number of steps ±y for the values m1˜n that have been saved in the memory. Here, M={mn±y×S|n being a natural number equal to or smaller than X, and y being an integer equal to or greater than 0 and having a maximum greater than 0}. The step interval S is determined by parameters for time-frequency expansion. The values m1˜n being used are the initial value m1=0 or the value that was saved in the memory at time Th−1 immediately preceding time Th as described later. This set M represents a set of allowable pulse rate values at the times from Th−1 to Th.

Then, in step S308, the signal processing unit 307 obtains a common set F∩M of the set F and the set M. The elements of this common set represent pulse rate candidates that fall within the range of allowable pulse rates. If F∩M=0 (yes in step S309), the elements of F are saved in working memories m1 to mx (step S310); if n(F∩M)≥1 (no in step S309), the elements of F∩M are saved in the working memories m1 to mx (step S311).

Particularly, if n(F∩M)=1 (yes in step S312), the frequencies of the elements of F∩M are converted to pulse rates (step S313), which are output to the transmission and reception unit 309 or recorded into the data saving memory, for example. For conversion of a frequency (Hz) into a pulse rate (bpm), the value of the frequency is multiplied by 60. Finally, it is determined whether measurement is to end (step S314), and a loop is performed.

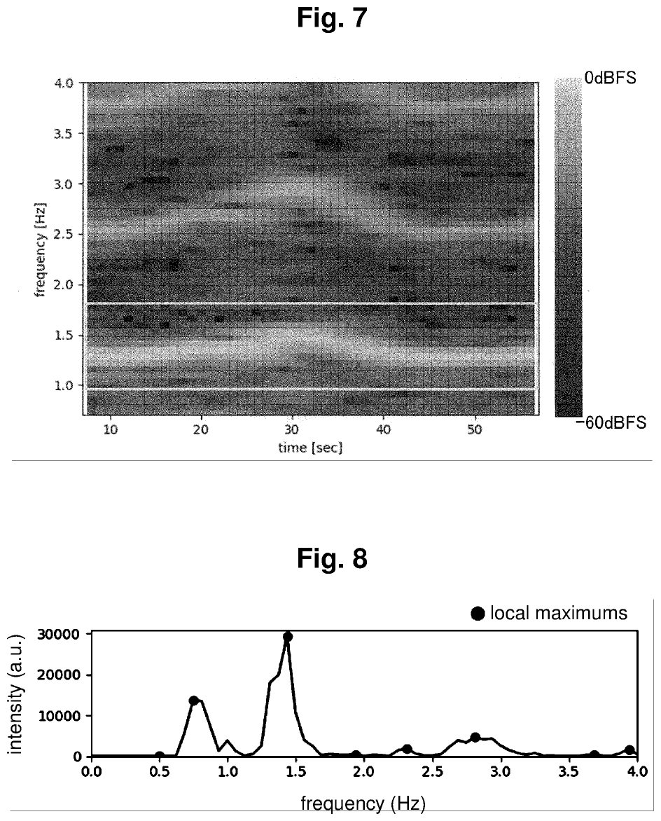

An example of actual measurement is now described using FIGS. 7, 8 and 9. FIG. 7 shows a spectrogram of a signal (pulse wave) observed over a certain time period, where the horizontal axis represents time, the vertical axis represents frequency, and the depth represents power spectrum. In this example, a bandpass filter was set at 0.7 to 4.0 Hz (42 to 240 bpm) based on the values at which the pulse rate was taken in order to emphasize the pulse rate. For the spectrogram parameters, sampling frequency was set at 64 Hz, a Hamming window was used as window function, window width was set at 16 s, and step was set at 1 s. As a pulse wave component appears as an emission line, change in the pulse rate can be followed by tracking the emission line. In FIG. 7, the emission line in the area surrounded by the square indicates variations in the pulse rate.

FIG. 8 shows a power spectrum at a certain time taken from spectrogram shown in FIG. 7, where the horizontal axis represents frequency and the vertical axis represents spectrum intensity. Local maximums that were detected in a peak search performed with the waveform shown in FIG. 6 are indicated by black circles. In this example, the interval of peak extraction was set to two points on each side.

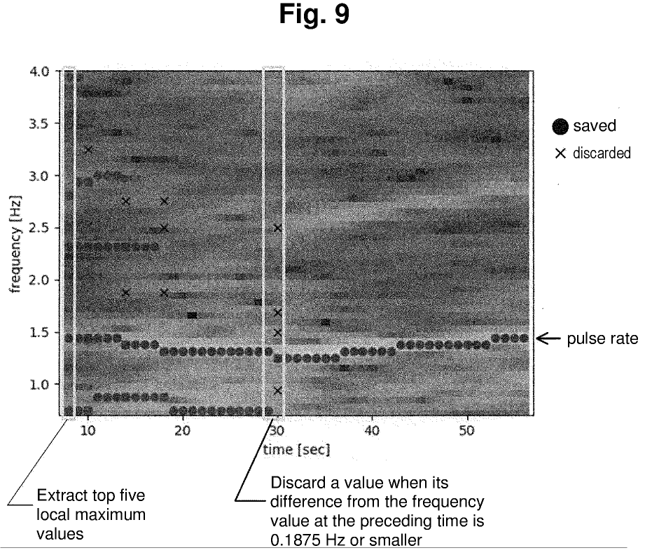

FIG. 9 shows a result of estimating the pulse rate on the spectrogram illustrated in FIG. 7. In this example, measurement was performed by the algorithm described with FIG. 6. Black circles on the spectrogram shown in FIG. 9 are points that were saved in the memory in the algorithm described with FIG. 6, while “x” marks indicate some of points that have been discarded without being saved. In the algorithm described with FIG. 6, X=5, the step interval S=0.1875 Hz, and y=2 were set as parameters. After 30 seconds, the pulse rate is narrowed down to one, from which point onwards the pulse rate is estimated. In FIG. 9, the area surrounded by the left square corresponds to the points that have been saved from the local maximums in the power spectrum shown in FIG. 8, and the area surrounded by the right square indicates a situation where values with a deviation from the frequency value at the preceding time being 0.1875 Hz or smaller are discarded.

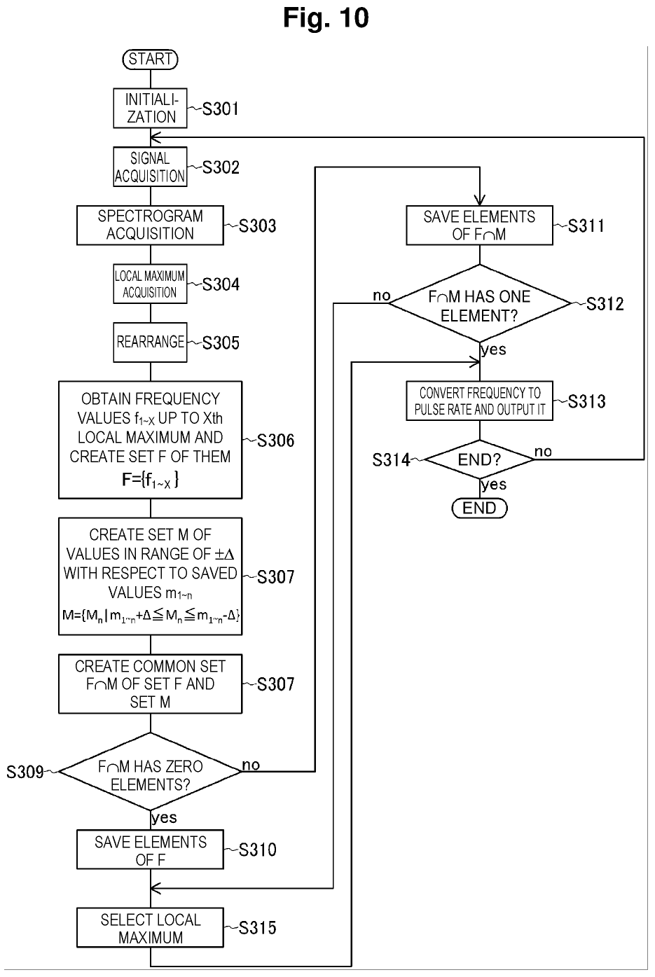

Next, another algorithm for determining the pulse rate from frequency components with a spectrogram (pulse rate estimation method) is described with reference to FIG. 10. First, in step S301, the working memories in the storage unit 308 are initialized. Here, x=0 and m1=0 are substituted into m1 to m2 saved in working memories. Then, in step S302, a pulse wave signal for the observed person is acquired for a certain period of time.

Specifically, upon the start, a particular light emitting element of the light emission unit 301 is caused to emit light at a predetermined light emission intensity through control by the light emission control unit 302 to irradiate the site of measurement 351. The incident light is scatted by arteriole near the site of measurement 351 to exit the skin surface at the site of measurement 351 and received by the light reception element(s) of the light reception unit 303. An analog signal corresponding to the amount of light thus received by the light reception unit 303 is output. The output signal is then amplified by the amplification unit 304 and is output to the signal processing unit 307 as an observation signal via the filter unit 305 and the A-D conversion unit 306.

Then, in step S303, the signal processing unit 307 performs time-frequency expansion such as short-time Fourier transform and wavelet transform based on chronological data for the observation signal (spectrogram acquisition).

Then, in step S304, the signal processing unit 307 obtains the power spectrum at a certain time Th, determines its local maximums by performing peak search on the obtained power spectrum, and records the intensity and frequency values of the local maximums. Specifically, a certain width is established on the frequency axis with respect to a function of the obtained spectrum and local maximums are determined within the established width. The local maximums are determined while shifting the width and the intensity and frequency values of all the local maximums are recorded.

Then, in step S305, the signal processing unit 307 sorts the recorded local maximums according to the power value to rearrange them in descending order. Then, in step S306, the signal processing unit 307 extracts up to the Xth local maximum and records their frequency values f1˜X. Here, the set of f1˜X is represented as F, where F={fX|X being a natural number}. These frequency values are candidates for the pulse rate.

Then, in step S307, the signal processing unit 307 creates a set M of values that fall within a range of a certain number of steps ±y for the values m1˜n that have been saved in the memory. Here, M={mn±y×S|n being a natural number equal to or smaller than X, and y being an integer equal to or greater than 0 and having a maximum greater than 0}. The step interval S is determined by parameters for time-frequency expansion. The values m1˜n being used are the initial value m1=0 or the value that was saved in the memory at time Th−1 immediately preceding time Th as described later. This set M represents a set of allowable pulse rate values at the times from Th−1 to Th.

Then, in step S308, the signal processing unit 307 obtains a common set F∩M of the set F and the set M. The elements of this common set represent pulse rate candidates that fall within the range of allowable pulse rates. If F∩M=0 (yes in step S309), the elements of F are saved in working memories m1 to mx (step S310), the element with the highest intensity among the elements saved in the working memories is selected (step S315), and the frequency of the selected element is converted to a pulse rate, which is output to the transmission and reception unit 309, for example (step S313). If n(F∩M)≥1 (no in step S309), the elements of F∩M are saved in working memories m1 to mx (step S311).

Particularly, if n(F∩M)=1 (yes in step S312), the frequencies of the elements of F∩M are converted to pulse rates (step S313). If n(F∩M)=1 does not hold (no in step S312), the element with the highest intensity among the elements saved in the working memories are selected (step S315), and the frequency of the selected element is converted to a pulse rate, which is output to the transmission and reception unit 309, for example (step S313). Finally, it is determined whether measurement is to end (step S314), and a loop is performed.

As described above, embodiments of the present invention perform time-frequency analysis on a detected pulse wave to determine a power spectrum for each time, determines local maximum points in each one of the power spectra determined for the respective times, and for each one of the power spectra determined for the respective times, extracts a certain number of largest values of the determined local maximum points as pulse rate candidates for that time. Embodiments of the present invention then compare a pulse rate candidate with a pulse rate candidate for the preceding time to determine a difference in frequency between them, and eliminate pulse rates for which the determined difference in frequency is equal to or greater than a preset reference value from the candidates. As a result, embodiments of the present invention can determine the pulse rate more accurately from the detected pulse wave.

Embodiments of the present invention employ time-frequency analysis for the estimation of the pulse wave. A feature of embodiments of the present invention is that it uses difference in spectrum between a pulse and body motion. The pulse rate is a periodical function and thus its spectrum contains peaks in the frequency domain, whereas body motion is nonperiodic noise and thus its spectrum tends to spread on the frequency axis. Accordingly, even during body motion, the pulse rate exhibits peaks in the frequency domain without being buried in the body motion. By determining such peaks, pulse rate candidates are determined.

A second feature of embodiments of the present invention is use of the constancy of the pulse rate. The pulse rate changes continuously because it acts to maintain constancy. By contrast, body motion exhibits an intermittent change because it occurs intermittently. These facts enable the pulse rate to be estimated even during motion by determining peaks that continuously vary on a spectrogram.

Embodiments of the present invention also use frequency components and intensity components in a spectrogram for the estimation of the pulse rate. By using frequency components and intensity components, embodiments of the present invention allow the pulse rate to be estimated at an earlier time, particularly in resting state. In resting state, spectral peaks are emphasized because there is little body motion. Thus, the frequency component of the local maximum having the highest spectral intensity can be estimated to be the pulse rate. Once the pulse rate has been estimated, the pulse rate can be determined by tracking the change in the frequency components, which thus enables the pulse rate to be determined even during motion. As embodiments of the present invention thus determine a single candidate for the pulse rate using frequency components and intensity components, it provides improved real-timeliness.

It is noted that the present invention is not limited to the above described embodiments but it is apparent that many variants and combinations are may be implemented by ordinary skilled persons in the art without departing from the technical scope of the present invention.

### REFERENCE SIGNS LIST

101 detection unit
102 first processing unit
103 second processing unit
104 third processing unit
105 fourth processing unit
106 fifth processing unit
107 storage unit
108 display unit.

1.-7. (canceled)

8. A pulse rate estimation method comprising:
chronologically detecting a pulse wave by receiving transmitted light that has been transmitted through an artery or scattered light that has been scattered by an artery; and
for each time period of a plurality of time periods corresponding to the pulse wave:
performing time-frequency analysis on the pulse wave to determine a power spectrum of the time period;
determining local maximum points in the power spectrum;
extracting largest values of the local maximum points as pulse rate candidates; and
for each pulse rate candidate of the pulse rate candidates, comparing the pulse rate candidate with a previous pulse rate candidate of a preceding time period to determine a difference in frequency and eliminating, from the pulse rate candidates, the pulse rate candidate when the difference in frequency being equal to or greater than a preset reference value.

9. The pulse rate estimation method according to claim 8, further comprising after eliminating, from the pulse rate candidates, the pulse rate candidate when the difference in frequency being equal to or greater than the preset reference value, determining a pulse rate candidate having a highest local maximum at each time period as an actual pulse rate.

10. The pulse rate estimation method according to claim 8, further comprising reducing noise in the pulse wave, wherein for each time period of the plurality of time periods, the time-frequency analysis is performed on the pulse wave with reduced noise.

11. A pulse rate estimation apparatus comprising:
a detector that chronologically detects a pulse wave by receiving transmitted light that has been transmitted through an artery or scattered light that has been scattered by an artery;
one or more processors; and
a non-transitory computer-readable storage medium storing a program to be executed by the one or more processors, the program including instructions to:
for each time period of a plurality of time periods corresponding to the pulse wave:
perform time-frequency analysis on the pulse wave to determine a power spectrum of the time period;
determine local maximum points in the power spectrum;
extract largest values of the local maximum points as pulse rate candidates; and
for each pulse rate candidate of the pulse rate candidates, compare the pulse rate candidate with a previous pulse rate candidate of a preceding time period to determine a difference in frequency and eliminate, from the pulse rate candidates, the pulse rate candidate when the difference in frequency being equal to or greater than a preset reference value.

12. The pulse rate estimation apparatus according to claim 11, wherein the instructions include further instructions to after eliminating, from the pulse rate candidates, the pulse rate candidate when the difference in frequency being equal to or greater than the preset reference value, determine a pulse rate candidate having a highest local maximum at each time period as an actual pulse rate.

13. The pulse rate estimation apparatus according to claim 11, further comprising a filter that reduces noise in the pulse wave detected by the detector, wherein the time-frequency analysis is performed on the pulse wave with reduced noise.

14. A non-transitory computer-readable media storing computer instructions for pulse rate estimation, that when executed by one or more processors, cause the one or more processors to perform the steps of:
chronologically detecting a pulse wave by receiving transmitted light that has been transmitted through an artery or scattered light that has been scattered by an artery; and
for each time period of a plurality of time periods corresponding to the pulse wave:
performing time-frequency analysis on the pulse wave to determine a power spectrum of the time period;
determining local maximum points in the power spectrum;
extracting largest values of the local maximum points as pulse rate candidates; and
for each pulse rate candidate of the pulse rate candidates, comparing the pulse rate candidate with a previous pulse rate candidate of a preceding time period to determine a difference in frequency and eliminating, from the pulse rate candidates, the pulse rate candidate when the difference in frequency being equal to or greater than a preset reference value.

15. The non-transitory computer-readable media of claim 14, wherein the instructions when executed by one or more processors, further cause the one or more processors to perform the steps of after eliminating, from the pulse rate candidates, the pulse rate candidate when the difference in frequency being equal to or greater than the preset reference value, determining a pulse rate candidate having a highest local maximum at each time period as an actual pulse rate.

16. The non-transitory computer-readable media of claim 14, wherein the instructions when executed by one or more processors, further cause the one or more processors to perform the steps of reducing noise in the pulse wave, wherein the time-frequency analysis is performed on the pulse wave with reduced noise.
